(19) United States
(12) Patent Application Publication
(10) Pub. No.: US 2024/0261547 A1
(43) Pub. Date: Aug. 8, 2024

(54) ANTIREFLUX URETERAL STENT AND MANUFACTURING METHOD FOR THE SAME

(71) Applicant: IUCF-HYU (INDUSTRY-UNIVERSITY COOPERATION FOUNDATION HANYANG UNIVERSITY), Seoul (KR)

(72) Inventors: Hongyun SO, Seoul (KR); Jung Ki JO, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 18/442,724

(22) Filed: Feb. 15, 2024

(30) Foreign Application Priority Data
Feb. 7, 2023 (KR) 10-2023-0016288
Sep. 5, 2023 (KR) 10-2023-0117337

(51) Int. Cl. A61M 27/00

(52) U.S. Cl. CPC A61M 27/008; A61M 2205/0216; A61M 2205/3331; A61M 2207/00; A61M 2210/1082; A61M 2210/1085

(57) ABSTRACT

Provided is an antireflux ureteral stent, including: a hollow tube-shaped stent body inserted into the ureter to guide urine from the kidneys to the bladder and provided with a flow path for flow of the urine thereinside; and an extratube backflow prevention mechanism provided in an umbrella shape, made of a flexible material, on an outer side surface of the stent body to be unfolded or folded along a flow direction of the urine and configured to prevent backflow of the urine while being unfolded by flow pressure of the urine when the urine backflows along an extratube gap formed between the stent body and the ureter, wherein the extratube backflow prevention mechanism is formed in a star-shaped cross-section with a plurality of vertices centered on the stent body and provided with a star-shaped cross section that expands toward the bladder along a longitudinal direction of the stent body.

### CROSS-REFERENCE TO RELATED APPLICATION

This application claims the priority benefit of Korean Patent Application No. 10-2023-0016288, filed on Feb. 7, 2023, and Korean Patent Application No. 10-2023-0117337, filed on Sep. 5, 2023, in the Korean Intellectual Property Office, the disclosure of each of which is incorporated herein by reference.

### BACKGROUND OF THE DISCLOSURE

### Field of the Disclosure

The present disclosure relates to an antireflux ureteral stent for preventing the reflux of urine from the bladder to the kidneys and a method of manufacturing the same, and more particularly to an antireflux ureteral stent having an optimized design structure to effectively prevent the reflux of urine between the ureter and an antireflux ureteral stent and increase urine reflux prevention performance and a method of manufacturing the antireflux ureteral stent.

### Description of the Related Art

Stents are widely used clinically to prevent stenosis of organs or blood vessels in the body, stenosis of the arteries, esophagus and gastrointestinal tract, stenosis of the biliary tract, stenosis of the ureter, etc. These stents can be inserted into areas, where flow is poor, to solve problems that impede the flow of fluids, such as blood, and various enzymes in blood vessels, biliary tracts, ureters, etc.

In particular, in the case of a ureter stent to prevent stenosis of the ureter, it is difficult to use a metal stent due to the nature of the ureter, so a double-J stent made of flexible plastic is used.

Both ends of the double-J stent are rolled up in a ‘J’ shape, making it difficult to adhere to the inner wall of the bladder. As a result, there is a problem of vesicoureteral reflux (VUR), which is the reflux of urine along the outer wall of the double-J stent. The vesicoureteral reflux problem can cause urine to flow back into the kidneys, causing a decrease in the function of the kidneys or causing pyelonephritis and hydronephrosis.

Recently, various technologies have been researched and developed to solve the problem of vesicoureteral reflux that urine flows back through a gap between the ureter and a stent.

### SUMMARY OF THE DISCLOSURE

Therefore, the present disclosure has been made in view of the above problems, and it is an object of the present disclosure to provide an antireflux ureteral stent capable of effectively preventing the vesicoureteral reflux (VUR) problem that urine backflows along a gap between the ureter and an antireflux ureteral stent; and a method of manufacturing the antireflux ureteral stent.

It is another object of the present disclosure to provide an antireflux ureteral stent on an outer side surface of a stent body of which an extratube backflow prevention mechanism is disposed to prevent vesicoureteral reflux and the shape and arrangement structure of an extratube backflow prevention mechanism of which are optimized to increase urine reflux prevention performance; and a method of manufacturing the antireflux ureteral stent.

In accordance with an aspect of the present disclosure, the above and other objects can be accomplished by the provision of an antireflux ureteral stent, including: a hollow tube-shaped stent body inserted into ureter to guide urine from kidneys to bladder and provided with a flow path for flow of the urine thereinside; and an extratube backflow prevention mechanism provided in an umbrella shape, made of a flexible material, on an outer side surface of the stent body to be unfolded or folded along a flow direction of the urine and configured to prevent backflow of the urine while being unfolded by flow pressure of the urine when the urine backflows along an extratube gap formed between the stent body and the ureter.

The extratube backflow prevention mechanism may be formed in a star-shaped cross-section with a plurality of vertices centered on the stent body and provided with a star-shaped cross section that expands toward the bladder along a longitudinal direction of the stent body.

Preferably, the size of the star-shaped cross section of the extratube backflow prevention mechanism may be reduced while urine passages for passing the urine are formed between the vertices of the star-shaped cross section as spaces between the vertices of the star-shaped cross section are folded into a furrow shape when a flow direction of the urine is a forward direction, and may increase while the urine passages formed between the vertices of the star-shaped cross section are removed as spaces between the vertices of the star-shaped cross section expand and spread when a flow direction of the urine is a reverse direction.

Preferably, the extratube backflow prevention mechanism may include: a fixation member fixed to an outer side surface of the stent body; a plurality of support beam members configured to expand long from the fixation member to the bladder to form the vertices of the star-shaped cross section of the stent body and formed in an inclined structure far away from the stent body as approaching the bladder; and a plurality of canopy members respectively provided between the support beam members to shield spaces between the support beam members.

Here, the extratube backflow prevention mechanism may be detachably mounted on an outer side surface of the stent body. Here, the fixation member may be formed in a tubular shape surrounding the outer side surface of the stent body. Accordingly, an installation groove for inserting and mounting the fixation member may be provided in a recessed structure along a perimeter of the outer side surface of the stent body.

In a different way from those described above, the extratube backflow prevention mechanism may be integrally formed with the stent body. Here, the fixation member may be connected to the outer side surface of the stent body in an integrated structure.

Preferably, the support beam members may be arranged to be spaced apart from each other at regular intervals along a perimeter of the fixation member and formed to be thicker than the canopy member to stably support the canopy members. The canopy members may be folded in a furrow shape toward the stent body according to flow pressure of the urine when a flow direction of the urine is a forward direction, and may be unfolded while expanding in an opposite direction of the stent body according to flow pressure of the urine when the flow direction of the urine is a reverse direction.

The support beam members and the canopy members may be formed in a funnel structure expanding along a longitudinal direction of the stent body toward the bladder side to form a urine inlet, into which the urine is introduced when the urine flows back, in an always open state.

Meanwhile, five support beam members may be provided to radially extend from the fixation member. Here, the canopy members may be respectively disposed between the support beam members and provided in a shape convexly curved toward the stent body such that they can be folded toward the outer side surface of the stent body.

Preferably, a bladder-side end of the stent body may be disposed inside one end of the ureter connected to the bladder, and a kidney-side end of the stent body may be disposed inside another end of the ureter connected to the kidneys. Here, at least one extratube backflow prevention mechanism may be disposed on the bladder-side end of the stent body.

Meanwhile, a single extratube backflow prevention mechanism may be disposed at a part connected to the bladder in the bladder-side end of the stent body.

Preferably, the extratube backflow prevention mechanism may be manufactured in an integrated structure through a casting process using a mold and a die, and the mold and the die may be manufactured through a 3D printing process.

Here, a cavity may be formed inside the mold and the die in a shape corresponding to the extratube backflow prevention mechanism, and a fine concavo-convex pattern corresponding to a stacking pattern of filaments may be formed on a surface of the cavity in a process of manufacturing the mold and the die through a 3D printing process.

In addition, a roughness pattern corresponding to the fine concavo-convex pattern may be formed on a surface of the extratube backflow prevention mechanism in a casting process using the mold and the die.

In accordance with another aspect of the present disclosure, there is provided a method of manufacturing an antireflux ureteral stent, the method including: manufacturing a mold and a die used in a casting process of an extratube backflow prevention mechanism using a 3D printing process; and manufacturing the extratube backflow prevention mechanism according to a casting process using the mold and the die.

Preferably, the manufacturing of the extratube backflow prevention mechanism may include: pouring a liquid elastomer material into the mold; coupling the die to the mold after degassing the elastomer material; thermally curing the elastomer material disposed in a cavity between the mold and the die with a heater; immersing the extratube backflow prevention mechanism, the mold and the die in an immersion liquid to peel the extratube backflow prevention mechanism from a surface of the cavity when the elastomer material is cured to form the extratube backflow prevention mechanism; taking the extratube backflow prevention mechanism, the mold and the die out of the immersion liquid, and then separating the mold and the die: and removing the extratube backflow prevention mechanism from an inside of the mold to complete manufacturing of the extratube backflow prevention mechanism.

Here, ecoflex may be provided as the elastomer material, and acetone may be provided as the immersion liquid. In addition, in the thermally heating of the heater, the elastomer material may be heated at 45 to 50° C. for 15 to 25 minutes with the heater to cure the elastomer material, and in the immersing of the immersion liquid, the extratube backflow prevention mechanism, the mold and the die may be immersed in the immersion liquid for 10 to 14 hours.

Preferably, in the manufacturing of the mold and the die, the mold and the die may be respectively manufactured using filaments made of polylactic acid (PLA) with a 3D printer by 3D printing method.

A fine concavo-convex pattern may be formed according to a stacking pattern of the filaments on surfaces of the mold and the die. In the manufacturing of the extratube backflow prevention mechanism, a roughness pattern may be formed on a surface of the extratube backflow prevention mechanism in a shape corresponding to the fine concavo-convex pattern of the mold and the die.

### BRIEF DESCRIPTION OF THE DRAWINGS

The above and other objects, features and other advantages of the present disclosure will be more clearly understood from the following detailed description taken in conjunction with the accompanying drawings, in which:

FIG. 1 illustrates the installation state of an antireflux ureteral stent according to an embodiment of the present disclosure;

FIG. 2 illustrates the antireflux ureteral stent of FIG. 1;

FIG. 3 illustrates a sectional view of a main part of the antireflux ureteral stent of FIG. 2;

FIG. 4 illustrates various types of the extratube backflow prevention mechanism shown in FIG. 2;

FIG. 5 illustrates the surface of the extratube backflow prevention mechanism shown in FIG. 4;

FIGS. 6 and 7 illustrate the results of measuring the performance of the extratube backflow prevention mechanism shown in FIG. 4, FIG. 6 illustrates a result measured when urine flows forward, and FIG. 7 illustrates a result measured when urine flows back;

FIG. 8 illustrates a method of manufacturing the antireflux ureteral stent according to an embodiment of the present disclosure;

FIG. 9 illustrates an example of installation of the antireflux ureteral stent shown in FIG. 8;

FIG. 10 schematically illustrates a performance test device of the antireflux ureteral stent according to an embodiment of the present disclosure;

FIG. 11 illustrates the antireflux performance of the antireflux ureteral stent dependent upon the number and installation positions of the extratube backflow prevention mechanisms using the performance test device shown in FIG. 10;

FIG. 12 illustrates the antireflux performance of an antireflux ureteral stent dependent upon the type and installation position of the extratube backflow prevention mechanism using the performance test device shown in FIG. 10; and

FIG. 13 illustrates displacement and acting force dependent upon the surface roughness of the extratube backflow prevention mechanism according to an embodiment of the present disclosure.

### DETAILED DESCRIPTION OF THE DISCLOSURE

Hereinafter, embodiments according to the present disclosure will be described in detail with reference to the attached drawings. However, the present disclosure is not limited by the embodiments. Identical reference numerals in each drawing indicate identical elements.

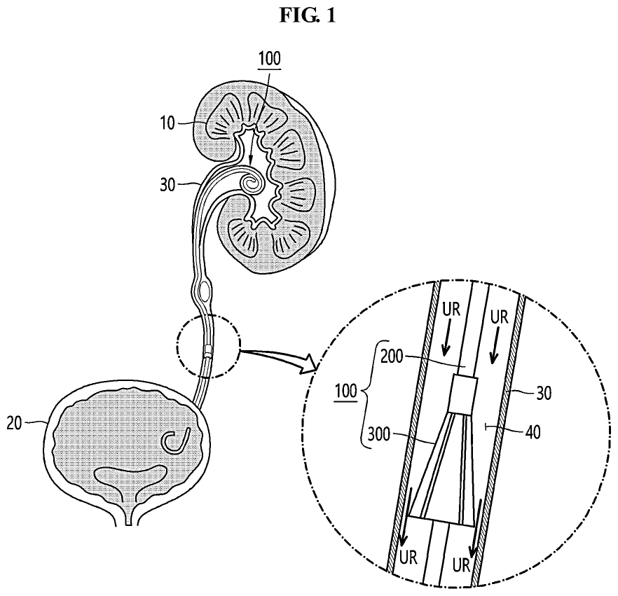

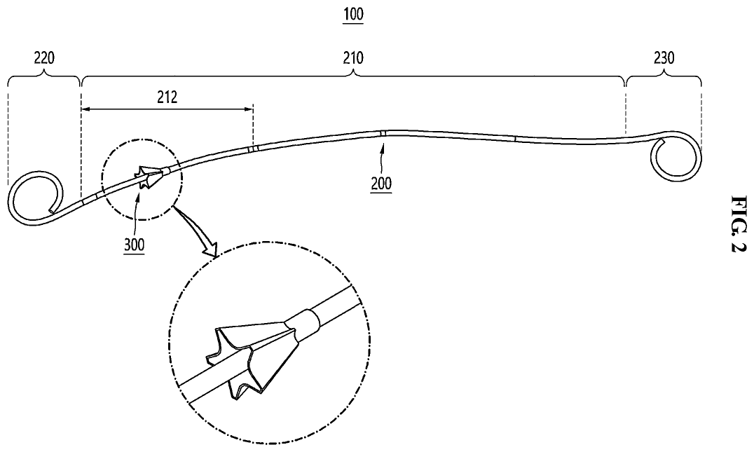

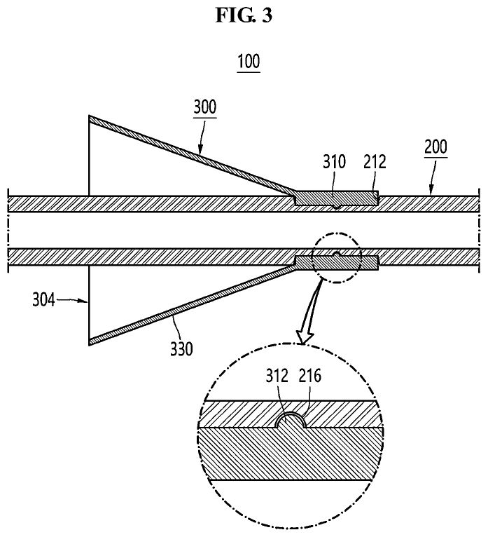

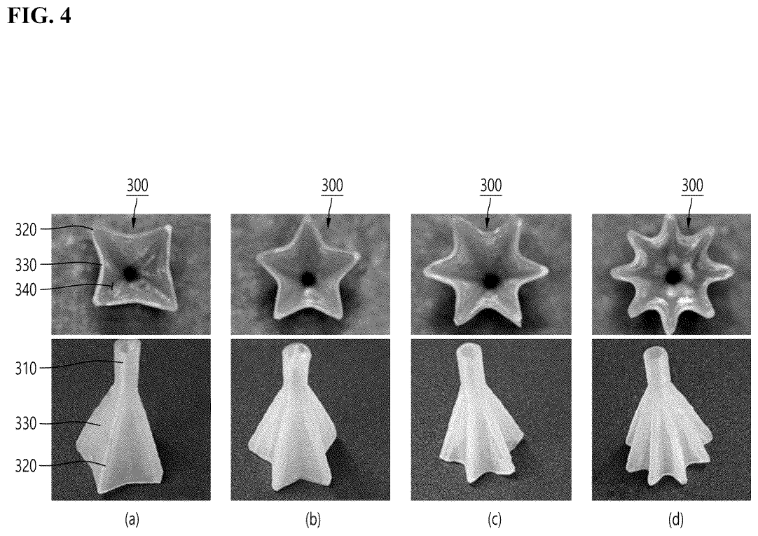

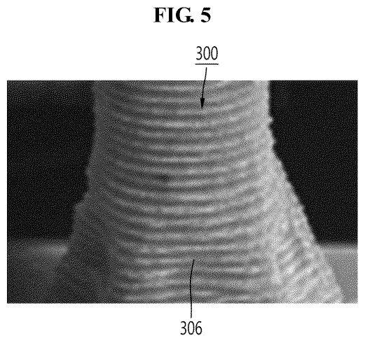

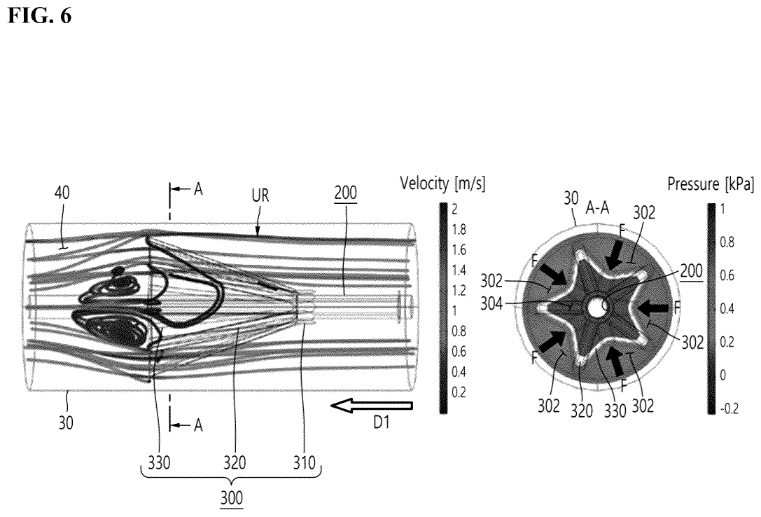

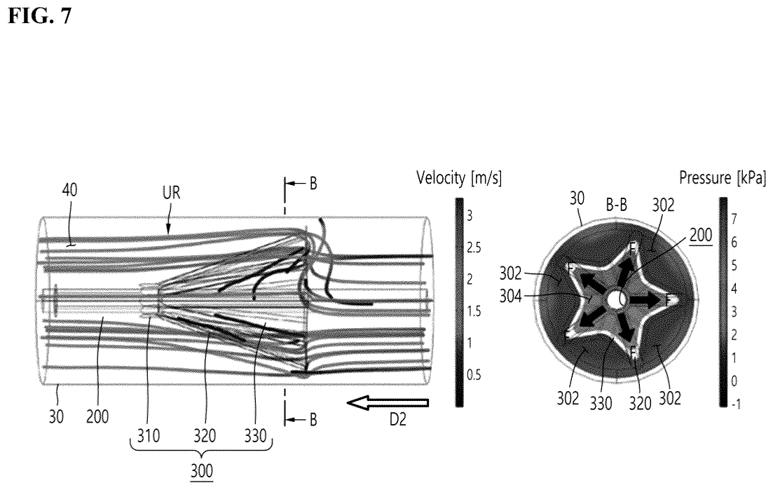

FIG. 1 illustrates the installation state of an antireflux ureteral stent 100 according to an embodiment of the present disclosure, FIG. 2 illustrates the antireflux ureteral stent 100 of FIG. 1, and FIG. 3 illustrates a sectional view of a main part of the antireflux ureteral stent 100 of FIG. 2. In addition, FIG. 4 illustrates various types of the extratube backflow prevention mechanism 300 shown in FIG. 2, and FIG. 5 illustrates the surface of the extratube backflow prevention mechanism 300 shown in FIG. 4. In addition, FIGS. 6 and 7 illustrate the results of measuring the performance of the extratube backflow prevention mechanism 300 shown in FIG. 4. In particular, FIG. 6 illustrates a result measured when urine flows forward, and FIG. 7 illustrates a result measured when urine flows back.

Referring to FIGS. 1 to 3, the antireflux ureteral stent 100 according to an embodiment of the present disclosure may include a stent body 200 and the extratube backflow prevention mechanism 300.

The antireflux ureteral stent 100 of this embodiment may be inserted into and placed inside the ureter 30 so that urine UR generated in the kidneys 10 can flow toward the bladder 20. In the following embodiment, the antireflux ureteral stent 100 is described as being provided as a double-J stent having both ends rolled in a ‘J’ shape.

In addition, the extratube backflow prevention mechanism 300 of this embodiment may be disposed on the outer surface of the stent body 200 to surround the outer surface of the stent body 200, and may be formed to protrude at a height that does not interfere with the inner wall of the ureter 30. The extratube backflow prevention mechanism 300 may selectively open/close an extratube gap 40, formed between the inner wall of the ureter 30 and the outer side surface of the stent body 200, along the flow direction of urine UR.

Referring to FIGS. 1 to 3, the stent body 200 of this embodiment may perform a function of guiding urine (UR) from the kidney 10 to the bladder 20. The stent body 200 may be formed in the shape of a hollow tube in which a flow path for the flow of urine (UR) is provided.

For example, the stent body 200 of this embodiment may include a body part 210, a bladder insertion part 220, and a kidney insertion part 230.

The body part 210 may be inserted inside the ureter 30. A bladder-side end of the body part 210 may be disposed inside one end of the ureter 30 connected to the bladder 20, and a kidney-side end of the body part 210 may be disposed inside the other end of the ureter 30 connected to the kidneys 10.

Here, an installation area 212 for installing the extratube backflow prevention mechanism 300 may be set at the bladder-side end of the body part 210, and at least one extratube backflow prevention mechanism 300 may be installed at the installation area 212. In the following embodiment, the installation area 212 may be positioned as close as possible to the bladder 20 side, and a single extratube backflow prevention mechanism 300 may be placed as close as possible to the bladder 20 side in the installation area 212.

The bladder insertion part 220 may have a structure rolled into a ‘J’ shape and may be connected to one end of the body part 210 to communicate therewith. The bladder insertion part 220 may be inserted inside the bladder 20. The bladder insertion part 220 may include an outlet for discharging urine UR flowing along the internal flow path of the body part 210 into the inside of the bladder 20.

The kidney insertion part 230 may have a structure rolled into a ‘J’ shape and connected to the other end of the body part 210 to communicate therewith. The kidney insertion part 230 may be inserted into the kidneys 10. The kidney insertion part 230 may include an inlet for supplying urine UR to the internal flow path of the body part 210.

Referring to FIGS. 1 to 7, the extratube backflow prevention mechanism 300 of this embodiment may be provided in an umbrella shape, made of a flexible material, on the outer side surface of the stent body 200 such that it can be unfolded or folded along the flow direction of urine UR. Here, the extratube backflow prevention mechanism 300 is unfolded by the flow pressure F of urine UR when urine UR flows back along the extratube gap 40 formed between the stent body 200 and the ureter 30, thereby preventing backflow of urine UR. For reference, the extratube backflow prevention mechanism 300 may be integrally formed on the outer peripheral surface of the installation area 212 of the stent body 200, and may be processed and molded together with the stent body 200 when manufacturing the stent body 200. However, in this embodiment, the structure wherein the extratube backflow prevention mechanism 300 and the stent body 200 are manufactured through separate processes, and then the extratube backflow prevention mechanism 300 is mounted on the outer side surface of the installation area 212 of the stent body 200 is described.

In addition, the extratube backflow prevention mechanism 300 may have a star-shaped cross section with a plurality of vertices centered on the stent body 200. The star-shaped cross section of the extratube backflow prevention mechanism 300 may be provided in a shape that expands toward the bladder 20 side along the longitudinal direction of the stent body 200.

Meanwhile, the extratube backflow prevention mechanism 300 of this embodiment may be manufactured as an integrated structure through a casting process using a mold 400 and a die 410. Preferably, the mold 400 and the die 410 of this embodiment may be be produced by stacking filaments into a three-dimensional structure through a 3D printing process.

Here, a cavity with a shape corresponding to the extratube backflow prevention mechanism 300 may be provided in the form of a clearance between the mold 400 and the die 410. A fine concavo-convex pattern may be formed on the surface of the cavity of the mold 400 and the die 410 to correspond to the stacking pattern of filaments according to the 3D printing process. Accordingly, a roughness pattern 306 corresponding to the fine concavo-convex pattern may be formed on the surface of the extratube backflow prevention mechanism 300 in the casting process using the mold 400 and the die 410.

With regard to this, FIG. 5 illustrates an enlarged photo of the surface of the extratube backflow prevention mechanism 300. Here, the roughness pattern 306 formed on the surface of the extratube backflow prevention mechanism 300 can be observed. The roughness pattern 306 may increase the surface roughness of the extratube backflow prevention mechanism 300. Accordingly, when the extratube backflow prevention mechanism 300 is mounted on the outer side surface of the stent body 200, the coupling force bettween the extratube backflow prevention mechanism 300 and the stent body 200 increases, effectively suppressing the separation of the extratube backflow prevention mechanism 300.

As shown in FIGS. 3 to 7, the extratube backflow prevention mechanism 300 may include a fixation member 310, a support beam member 320 and a canopy member 330.

The fixation member 310 may be fixed to the outer side surface of the stent body 200. For this, the fixation member 310 may be formed in a tubular shape surrounding the outer side surface of the stent body 200.

As shwon in FIG. 3, an installation groove 214 for inserting and mounting the fixation member 310 may be provided in a recessed structure along the circumference in the installation area 212 of the outer side surface of the stent body 200, and a protruding structure for insertion into the installation groove 214 may be provided on the inner peripheral surface of the fixation member 310. Here, a position-fixing protrusion 312 may be formed on one of the inner peripheral surface of the fixation member 310 and the bottom surface of the installation groove 214, and a position-fixing groove 216 for insertion of the position-fixing protrusion 312 may be formed on the other one thereof to correspond to the position-fixing protrusion 312.

In the following embodiment, a plurality of position-fixing protrusions 312 may be formed on the inner peripheral surface of the fixation member 310, and a plurality of position-fixing grooves 216 may be formed on the bottom surface of the installation groove 214 to correspond to the position-fixing protrusions 312. Accordingly, the fixation member 310 of the extratube backflow prevention mechanism 300 may be exactly installed at a predetermined exact position in the installation area 212 of the stent body 200 by engaging the position-fixing protrusion 312 and the position-fixing groove 216 after being inserted into and mounted in the installation groove 214. Accordingly, it is possible to stably prevent arbitrary movement or removal from the outer side surface of the stent body 200 due to external shock or vibration.

A plurality of the support beam members 320 may extend long from the fixation member 310 to the bladder 20 side to form vertices of the star-shaped cross section of the stent body 200. The support beam members 320 may be arranged to be spaced apart at regular intervals along the perimeter of the fixation member 310. In addition, the support beam member 320 may be formed in an inclined structure that moves away from the stent body 200 toward the bladder 20 side. In addition, the support beam member 320 may be formed to be thicker than the canopy member 330 to stably support the canopy member 330.

The canopy members 330 may be respectively provided between the support beam members 320 to shield the space between the support beam members 320. When the flow direction of urine UR is a forward direction D1, the canopy members 330 may be folded in a furrow shape toward the stent body 200 along the flow pressure F of urine UR, and when the flow direction of urine UR is a reverse direction D2, the canopy members 330 may be unfolded while expending toward the opposite direction of the stent body 200 along the flow pressure F of urine UR.

Meanwhile, the support beam members 320 and the canopy members 330 may form a urine inlet 304 in an always open state for inflow of urine UR when the urine UR flows back, and may form the urine inlet 304 in a funnel structure extending along the longitudinal direction of the stent body 200 toward the bladder 20 side. For example, five support beam members 320 may be provided to extend radially from the fixation member 310, and the canopy members 330 may be respectively arranged between the support beam members 320. The canopy members 330 may be provided in a convexly curved shape toward the stent body 200 such that they can be folded toward the outer side surface of the stent body 200.

FIG. 4 illustrates various examples of extratube backflow prevention mechanisms 300 classified according to a star-shaped cross section shape.

As shown in FIG. 4, the extratube backflow prevention mechanism 300 may have various star-shaped cross sections depending on the number and arrangement structure of the support beam members 320 and the canopy members 330. Here, the fixation member 310, the support beam members 320 and the canopy members 330 may be manufactured in an integrated structure according to a casting process. The fixation member 310 may be provided to be located at the center of the star-shaped cross section of the extratube backflow prevention mechanism 300, the support beam members 320 may be provided to form the vertices of the star-shaped cross section of the extratube backflow prevention mechanism 300, and the canopy members 330 may be provided to connect the vertices of the star-shaped cross section of the extratube backflow prevention mechanism 300.

Here, FIG. 4A illustrates a quadra-type extratube backflow prevention mechanism 300 formed to have a star-shaped cross section with four vertices. The quadra-type extratube backflow prevention mechanism 300 is composed of one fixation member 310, four support beam members 320 and four canopy members 330. Here, the urine inlet 304 may also have a quadra-type star-shaped cross section.

In addition, FIG. 4B illustrates a penta-type extratube backflow prevention mechanism 300 formed to have a star-shaped cross section with five vertices. The penta-type extratube backflow prevention mechanism 300 is composed of one fixation member 310, five support beam members 320 and five canopy members 330. Here, the urine inlet 304 may also have a penta-type star-shaped cross section.

In addition, FIG. 4C illustrates a hexa-type extratube backflow prevention mechanism 300 formed to have a star-shaped cross section with six vertices. The hexa-type extratube backflow prevention mechanism 300 is composed of one fixation member 310, six support beam members 320 and six canopy members 330. Here, the urine inlet 304 may also have a hexa-type star-shaped cross section.

In addition, FIG. 4D illustrates an octa-type extratube backflow prevention mechanism 300 formed to have a star-shaped cross section with eight vertices. The octa-type extratube backflow prevention mechanism 300 is composed of one fixation member 310, eight support beam members 320 and eight canopy members 330. Here, the urine inlet 304 may also have an octa-type star-shaped cross section.

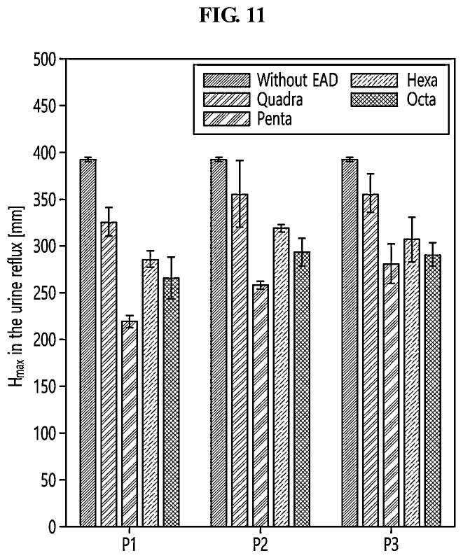

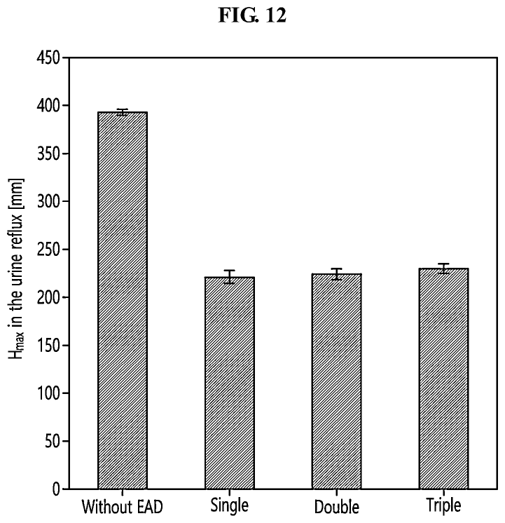

The extratube backflow prevention mechanisms 300 shown in FIG. 4 maybe classified depending upon the star-shaped cross section shapes formed by the support beam members 320 and the canopy members 330. That is, the space formed between the two support beam members 320 may become smaller as the number of the vertices in the star-shaped cross section increases, and, as a result, the size of the canopy members 330 may also be smaller and the range of being folded or unfolded may also be reduced. Accordingly, in the extratube backflow prevention mechanisms 300 shown in FIG. 4, the number of urine passage 302 increases as the number of the vertices in the star-shaped cross section increases. However, since the folding range of the canopy members 330 descreases, the size of the urine passages 302 may become smaller. The performance test results of the various extratube backflow prevention mechanisms 300 are illustrated through the graphs of FIGS. 11 and 12 below. So, a detailed description is provided below.

FIGS. 6 and 7 illustrate simulation results of the extratube backflow prevention mechanism 300 respectively measured along the flow direction of urine UR using the extratube backflow prevention mechanism 300 shown in FIG. 4B.

For reference, in the case of FIG. 6 where urine UR flows in the forward direction D1, the renal pelvic pressure is approximately 15˜18 cm-H₂ O hydrostatic pressure, so the inlet pressures of the stent body 200 and the extratube gap 40 of the ureter 30 are set to 18 cm-H₂ O(1765 Pa). In addition, in the case of FIG. 7 where urine UR flows in the reverse direction D2, the inlet pressures of the stent body 200 and the extratube gap 40 of the ureter 30 are set to 50 cm-H₂ O(4903 Pa) of an intravenous pressure relative to a void.

FIGS. 6 and 7 simulate pressure distribution observed around the extratube backflow prevention mechanism 300 during the forward direction D1 flow and reverse direction D2 flow of urine UR.

As shown in FIG. 6, in the extratube backflow prevention mechanism 300, the space between the vertices of the star-shaped cross section may be folded in a furrow shape by the flow pressure F of urine UR when the flow direction of urine UR is the forward direction D1. That is, the canopy members 330 between the support beam members 320 forming the vertices of the star-shaped cross section may be folded in a furrow shape, so that the overall size of the extratube backflow prevention mechanism 300 is reduced and the urine passages 302 may be respectively formed between the support beam members 320. The urine passage 302 is provided to serve as a passage for passing urine UR flowing in the forward direction D1, and may be formed in a slot shape between the inner wall of the ureter 30 and the folded canopy members 330.

As shown in FIG. 7, the extratube backflow prevention mechanism 300 may be unfolded such that the space between the vertices of the star-shaped cross section is expanded by the flow pressure F of urine UR when the flow direction of urine UR is the reverse direction D2. That is, the canopy members 330 present between the support beam members 320 forming the vertices of the star-shaped cross section expand and are unfolded, so that the overall size of the extratube backflow prevention mechanism 300 increases and the urine passages 302 between the support beam members 320 may be removed. The canopy members 330 may expand toward the inner wall of the ureter 30 to block the internal flow path of the ureter 30.

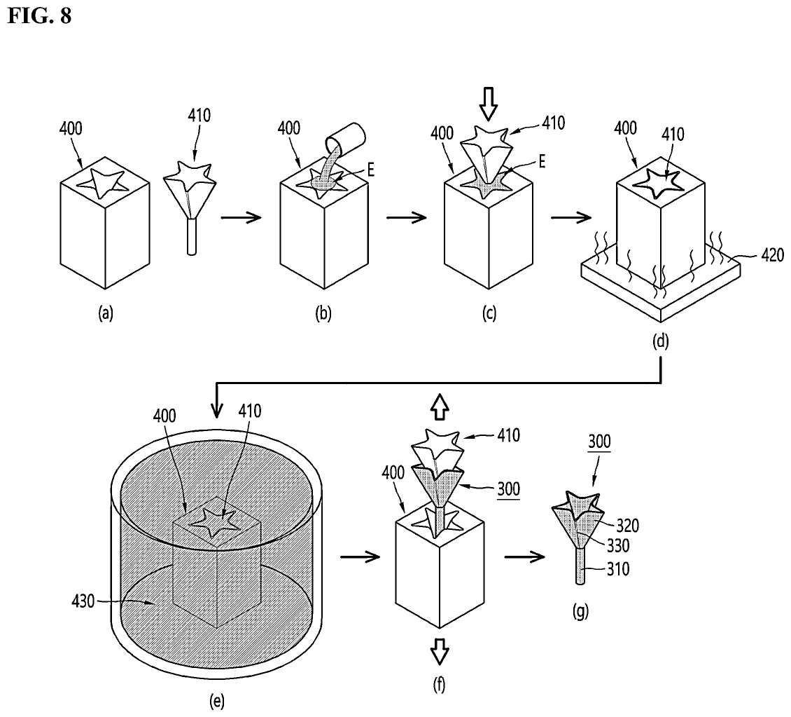

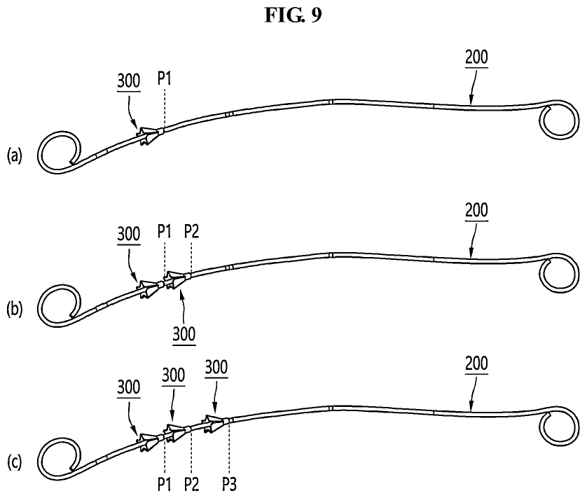

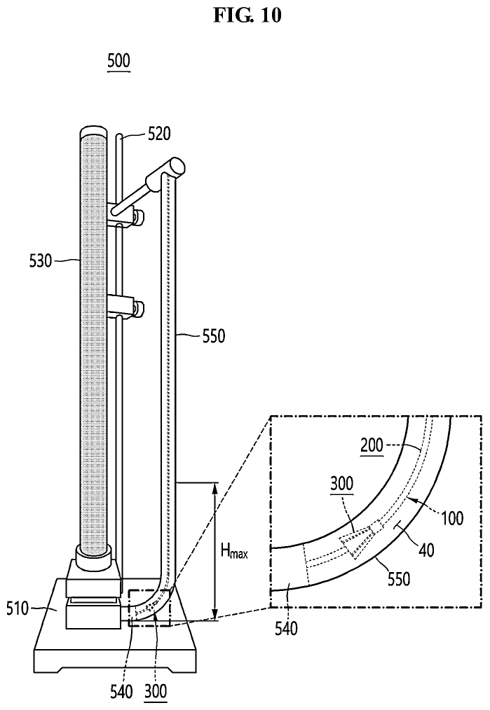

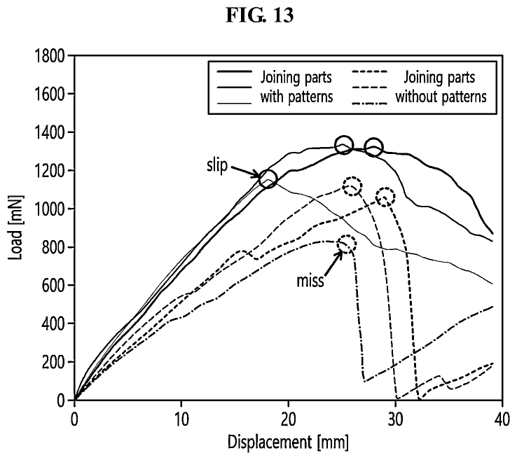

FIG. 8 illustrates a method of manufacturing the antireflux ureteral stent 100 according to an embodiment of the present disclosure. FIG. 9 illustrates an example of installation of the antireflux ureteral stent 100 shown in FIG. 8, and FIG. 10 schematically illustrates a performance test device 500 of the antireflux ureteral stent 100 according to an embodiment of the present disclosure. In addition, FIG. 11 illustrates the antireflux performance of the antireflux ureteral stent 100 dependent upon the number and installation positions of the extratube backflow prevention mechanisms 300 using the performance test device 500 shown in FIG. 10, FIG. 12 illustrates the antireflux performance of an antireflux ureteral stent dependent upon the type and installation position of the extratube backflow prevention mechanism 300 using the performance test device shown in FIG. 10, and FIG. 13 illustrates displacement and acting force dependent upon the surface roughness of the extratube backflow prevention mechanism 300 according to an embodiment of the present disclosure.

A method of manufacturing the antireflux ureteral stent 100 according to an embodiment of the present disclosure configured as described above and performance test results thereof are described below.

Referring to FIG. 8, the method of manufacturing the antireflux ureteral stent 100 according to an embodiment of the present disclosure may include a step of manufacturing the mold 400 and the die 410, used in a casting process of the extratube backflow prevention mechanism 300, using a 3D printing process (see FIG. 8A); and a step of manufacturing the extratube backflow prevention mechanism 300 according to the casting process using the mold 400 and the die 410 (see FIGS. 8B to 8G).

In the step of manufacturing the mold 400 and the die 410 (see FIG. 8A), the mold 400 and the die 410 are manufactured according to a 3D printing process using a 3D printer.

That is, in the step of manufacturing the mold 400 and the die 410 (see FIG. 8A), the mold 400 and the die 410 are respectively manufactured by a 3D printer using filaments made of polylactic acid (PLA) according to Fused Deposition Modeling (FDM) printing as a 3D printing method. Here, a cavity corresponding to the shape of the extratube backflow prevention mechanism 300 shown in FIG. 4 maybe formed between the die 410 and the mold 400. For example, this embodiment may provide the cavity of the die 410 and the mold 400 in the form of a clearance formed between the die 410 and the mold 400.

Accordingly, the mold 400 and the die 410 may be manufactured in various ways according to the shape of the star-shaped cross section of the extratube backflow prevention mechanism 300 by controlling the operation of a 3D printer. Hereinafter, a penta-type extratube backflow prevention mechanism 300 is described in this embodiment for convenience of explanation.

Meanwhile, the mold 400 and the die 410 may be used in the casting process of the extratube backflow prevention mechanism 300. Here, a fine concavo-convex pattern is formed on the surfaces of the mold 400 and the die 410 in a process of laminating filaments made of PLA according to a 3D printing technique. By the fine concavo-convex pattern of the mold 400 and the die 410, the roughness pattern 306 may be formed on the surface of the extratube backflow prevention mechanism 300 in a casting process of the extratube backflow prevention mechanism 300 described below.

In the step of manufacturing the extratube backflow prevention mechanism 300 (see FIGS. 8B to 8G), the extratube backflow prevention mechanism 300 is manufactured through a casting process using the mold 400 and the die 410.

As shown in FIG. 8, the step of manufacturing the extratube backflow prevention mechanism 300 (see FIGS. 8B to 8G) includes a step of pouring a liquid elastomer material E into the mold 400 (see FIG. 8B); a step of pouring a liquid elastomer material E into the mold 400 (see FIG. 8C); a step of heating and curing the elastomer material E disposed in a cavity between the mold 400 and the die 410 with a heater 420 (see FIG. 8D); a step of immersing the extratube backflow prevention mechanism 300, the mold 400 and the die 410 in an immersion liquid 430 to peel the extratube backflow prevention mechanism 300 from the surface of the cavity when the elastomer material E is cured to form the extratube backflow prevention mechanism 300 (see FIG. 8E); a step of taking the extratube backflow prevention mechanism 300, the mold 400 and the die 410 out of the immersion liquid 430, and then separaitng the mold 400 and the die 410 (see FIG. 8F); and a step of removing the extratube backflow prevention mechanism 300 from the inside of the mold 400 to complete the manufacture of the extratube backflow prevention device 300 (see FIG. 8G).

In the step of pouring the elastomer material E (see FIG. 8B), the liquid elastomer material E is injected into the cavity formed in the mold 400.

Here, the elastomer material E may selectively provide at least one of ecoflex, polydimethylsiloxane (PDMS), polyvinyl chloride (PVC), natural rubber, silicone, polymethly methacrylate (PMMA), polyurethane (PU), thermoplastic elastomer (TPE), ethylene-vinylacetate alcohol polymer (EVAL polymer), polypropylene (PP), polyethylene (PE), acetyl cellulose, polyethyleneterephthalate (PET), and polytetrafluoroethylene (PTFE).

In the step of coupling the die 410 to the mold 400 (see FIG. 8C), the elastomer material E is degassed, and then the die 410 is coupled to the mold 400.

Here, the degassing of the elastomer material E may be performed for 10 minutes, so that air bubbles trapped by the elastomer material E can be removed. When the degassing process of the elastomer material E is completed, the die 410 is coupled to the mold 400. Accordingly, a cavity having the same shape as the extratube backflow prevention mechanism 300 is formed in the form of a clearance between the die 410 and the mold 400.

In the step of heating and curing with the heater 420 (see FIG. 8D), the elastomer material E disposed in the cavity between the mold 400 and the die 410 is heated with the heater 420 to cure into the extratube backflow prevention mechanism 300.

Here, the heating temperature of the heater 420 should be set to a sufficiently high temperature to thermally cure the elastomer material E and to a lower temperature than the melting temperature of the mold 400 and the die 410. For reference, since the mold 400 and the die 410 are manufactured with filaments made of PLA in this embodiment, the heating temperature of the heater 420 should be set lower than 55 to 70° C. that is the glass transition temperature for the filaments made of PLA.

For example, in the step of heating and curing with the heater 420 (see FIG. 8D), the elastomer material E is cured by heating the elastomer material E contained in the cavity at 45 to 50° C. for 15 to 25 minutes with the heater 420. Preferably, the elastomer material E may be heated at 50° C. for 20 minutes with the heater 420 in this embodiment.

In the step of immersing in the immersion liquid E (see FIG. 8E), the extratube backflow prevention mechanism 300 cured by the heater 420 in the previous step is peeled off from the surface of the cavity of the die 410 and the mold 400 by immersing the extratube backflow prevention mechanism 300, the mold 400 and the die 410 in the immersion liquid 430. In this embodiment, acetone is provided as the immersion liquid 430.

In the following embodiment, the extratube backflow prevention mechanism 300, the mold 400 and the die 410 may be immersed in the immersion liquid 430 for 10 to 14 hours. Preferably, it is set to be immersed in the immersion liquid 430 for 12 hours in this embodiment.

In the step of separating the mold 400 and the die 410 (see FIG. 8F), the extratube backflow prevention mechanism 300, the mold 400 and the die 410 are taken out of the immersion liquid 430, and then the extratube backflow prevention mechanism 300 is exposed to the outside by separting the die 410 from the mold 400.

In the step of completing the manufacture of the extratube backflow prevention device 300 (see FIG. 8G), the extratube backflow prevention mechanism 300 is easily removed from the inside of the mold 400, thereby obtaining the extratube backflow prevention mechanism 300 manufactured through the casting process.

FIG. 9 illustrates installation examples of the extratube backflow prevention mechanism 300 of FIG. 8 in the installation area of the stent body 200, and FIG. 10 schematically illustrates the configuration of the performance test device 500 for testing the performance of the antireflux ureteral stent 100 shown in FIG. 9.

As shown in FIG. 9, at least one extratube backflow prevention mechanism 300 may be installed in the installation area 212 of the body part 210 of the stent body 200.

That is, FIG. 9A illustrates an antireflux ureteral stent 100 wherein one extratube backflow prevention mechanism 300 is installed at a first position P1 in the installation area 212 of the body part 210 of the stent body 200. Here, the first position P1 is a position in the installation area 212 closest to the bladder 20.

In addition, FIG. 9B illustrates an antireflux ureteral stent 100 wherein two extratube backflow prevention mechanisms 300 are installed at the first and second positions P1 and P2 in the installation area 212 of the body part 210 of the stent body 200. Here, the second position P2 may be provided at a position spaced apart from the first position P1 by a first set distance toward the kidneys 10.

In addition, FIG. 9C illustrates an antireflux ureteral stent 100 where three extratube backflow prevention mechanisms 300 are installed at the first, second and third positions P1, P2 and P3 in the installation area 212 of the body part 210 of the stent body 200. Here, the third position P3 may be positioned at a position spaced apart by a second set distance greater than the first set distance from the second position P2 toward the kidneys 10.

In this embodiment, the first set distance is set to 17.5 mm and the second set distance is set to 22 mm to perform performance experiments. In this embodiment, the extratube backflow prevention mechanisms 300 installed at the first position P1, the second position P2 and the third position P3 are set to be of the same penta-type.

As shown in FIG. 10, the performance test device 500 of this embodiment is an experimental device that simulates the bladder 20 and the ureter 30 and has antireflux performance for urine UR flowing back from the bladder 20. For example, the performance test device 500 of this embodiment may include a base 510, a stand 520, a bladder replica tube 530, a tube connection part 540, and a ureter replica tube 550.

Here, the base 510 is a component for supporting the performance test device 500, and the stand 520 is a component for supporting the bladder replica tube 530 and the ureter replica tube 550.

In addition, liquid is contained inside the bladder replica tube 530 to simulate flow in the reverse direction D2 of urine UR and may be formed in the shape of a vertically standing hollow tube to simulate the flow pressure of urine UR Here, it is also possible to install an air pressure generator at an upper part of the bladder replica tube 530 and set the pressure of the liquid discharged to a lower part of the bladder replica tube 530 through the operation of an air pressure generator, but, in this embodiment, the pressure of the liquid discharged to the lower part of the bladder replica tube 530 is set using the weight of the liquid filled inside the bladder replica tube 530.

In addition, one side part of the tube connection part 540 may be connected in communication with the lower part of the bladder replica tube 530, and the other side part of the tube connection part 540 may be connected in communication with the lower part of the ureter replica tube 550. The tube connection part 540 may play the role of guiding liquid discharged to the lower part of the bladder replica tube 530 to the ureter replica tube 550.

In addition, the ureter replica tube 550 may be formed in the shape of a hollow tube with an empty interior to simulate the ureter 30. The ureter replica tube 550 may be stably and vertically erected by the stand 520. The antireflux ureteral stent 100 may be placed inside the ureter replica tube 550.

Meanwhile, the performance test device 500 of this embodiment measures the antireflux performance of the antireflux ureteral stent 100 in a process of flowing the liquid stored in the bladder replica tube 530 through the ureter replica tube 550 via the tube connection part 540. Here, the antireflux performance of the antireflux ureteral stent 100 is evaluated by measuring the average maximum height (Hmax) of liquid flowing into the ureter replica tube 550. The antireflux performance is evaluated to be excellent as the average maximum height (Hmax) of the liquid is small.

FIG. 11 illustrates the antireflux performances dependent upon the number and installation positions of the extratube backflow prevention mechanisms 300 using the performance test device 500 shown in FIG. 10.

That is, the graphs of FIG. 11 show the antireflux performance analysis results of the antireflux ureteral stents 100 shown in FIG. 9 and an antireflux ureteral stent 100, in which the extratube backflow prevention mechanism 300 is not installed, using the performance test device 500 shown in FIG. 10.

Referring to FIG. 11, to compare the antireflux performances of the performance test devices 500, a state (Without EAD) in which the extratube backflow prevention mechanism 300 is not installed at the stent body 200, a state (Single) in which one extratube backflow prevention mechanism 300 is installed at the first position P1 of the stent body 200, a state (Double) in which two extratube backflow prevention mechanisms 300 are installed at the first and second positions P1 and P2 of the stent body 200, and a state (Triple) in which three extratube backflow prevention mechanisms 300 are installed at the first, second and third positions P1, P2 and P3 of the stent body 200 were analyzed.

As analysis results of the graphs of FIG. 11, the antireflux ureteral stent 100 (Without EAD) in which the extratube backflow prevention mechanism 300 is not installed at the stent body 200 exhibits the highest average maximum height (Hmax) of the liquid, thereby exhibiting the lowest antireflux performance.

On the other hand, as analysis results of the graphs of FIG. 11, the antireflux ureteral stents 100 (Single, Double, Triple) in which one to three extratube backflow prevention mechanisms 300 are respectively installed at the first to third positions P1, P2 and P3 of the stent body 200 exhibit almost the same average maximum heights (Hmax) of the liquid, which indicates that the antireflux performances are similar or the same. Accordingly, considering the antireflux performance of the antireflux ureteral stent 100, it can be seen that it is sufficient to install a single stent body 200 in the extratube backflow prevention mechanism 300.

FIG. 12 illustrates the antireflux performances dependent upon the types and installation positions of the extratube backflow prevention mechanisms 300 using the performance test device 500 shown in FIG. 10.

That is, the graphs of FIG. 12 show the antireflux performance analysis results of antireflux ureteral stents 100, in which a single extratube backflow prevention mechanism 300 shown in FIG. 4 is installed at each of the first to third positions P1, P2 and P3 of the stent body 200, using the performance test device 500 shown in FIG. 10.

Referring to FIG. 12, to compare the antireflux performances of the performance test devices 500, the antireflux ureteral stent 100 (Without EAD) in which the extratube backflow prevention mechanism 300 is not installed at the stent body 200, the antireflux ureteral stent 100 (Quadra) in which a single quadra-type extratube backflow prevention mechanism 300 is installed at each of the first to third positions P1, P2 and P3 of the stent body 200, the antireflux ureteral stent 100 (Penta) in which a single penta-type extratube backflow prevention mechanism 300 is installed at each of the first to third positions P1, P2 and P3 of the stent body 200, the antireflux ureteral stent 100 (Hexa) in which a single hexa-type extratube backflow prevention mechanism 300 is installed at each of the first to third positions P1, P2 and P3 of the stent body 200, and the antireflux ureteral stent 100 (Octa) in which a single octa-type extratube backflow prevention mechanism 300 is installed at each of the first to third positions P1, P2 and P3 of the stent body 200 were analyzed.

As analysis results of the graphs of FIG. 12, the antireflux ureteral stents 100 (Without EAD) in which the extratube backflow prevention mechanism 300 is not installed at the stent body 200 exhibit the highest and same average maximum height (Hmax) of liquid regardless the shape and installation position of the extratube backflow prevention mechanism 300, so the antireflux performance is analyzed to be the lowest.

On the other hand, as analysis results of the graphs of FIG. 12, the antireflux ureteral stents 100 (P1, P2, P3) in which only a single extratube backflow prevention mechanism 300 among the various types of extratube backflow prevention mechanisms 300 is installed at each of the first to third positions P1, P2 and P3 of the stent body 200 exhibit that the average maximum height (Hmax) of liquid is relatively high as the extratube backflow prevention mechanism 300 approaches the bladder 20 side. Accordingly, considering the antireflux performance of the antireflux ureteral stents 100, it can be seen that the antireflux performance is the best at the first position P1 closest to the bladder 20 side regardless the type of the extratube backflow prevention mechanism 300.

In addition, as analysis results of the graphs of FIG. 12, the antireflux ureteral stents 100 (Without EAD, Quadra, Penta, Hexa, Octa) in which various types of extratube backflow prevention mechanisms 300 are respectively installed at the first to third positions P1, P2 and P3 of the stent body 200 exhibit that the average maximum height (Hmax) of liquid is relatively high in the order of penta-type, octa-type, hexa-type and quad type. Accordingly, considering the antireflux performance of the antireflux ureteral stent 100, it can be seen that the penta-type extratube backflow prevention mechanism 300 exhibits the best antireflux performance compared to other types (octa-type, hexa-type and quad type).

FIG. 13 illustrates analysis results of the displacement and acting force dependent upon the surface roughness of the extratube backflow prevention mechanism 300 according to an embodiment of the present disclosure.

That is, the graphs of FIG. 13 illustrate displacement changes used to analyze slip or miss from the stent body 200 according to acting force (load) applied to the extratube backflow prevention mechanisms 300 using the extratube backflow prevention mechanisms 300 with or without the roughness pattern 306 shown in FIG. 5 on the surface thereof.

Referring to FIG. 13, it can be confirmed that the extratube backflow prevention mechanism 300 with the roughness pattern 306 on the surface thereof is slipped or missed at a higher acting force compared to the extratube backflow prevention mechanism 300 without the roughness pattern 306 on the surface thereof. Accordingly, it can be seen that slip or arbitrary removal of the extratube backflow prevention mechanism 300 from the outer side surface of the stent body 200 can be effectively prevented by forming the roughness pattern 306 on the surface of the extratube backflow prevention mechanism 300.

In acccordance with an antireflux ureteral stent according to an embodiment of the present disclosure and a method of manufacturing the same, the vesicoureteral reflux (VUR) problem that urine backflows along an extratube gap formed between the ureter and the antireflux ureteral stent can be easily and effectively prevented by disposing an extratube backflow prevention mechanism on an outer side surface of a stent body.

In acccordance with the antireflux ureteral stent according to an embodiment of the present disclosure and the method of manufacturing the same, the urine reflux prevention performance can be stably increased by designing the extratube backflow prevention mechanism in a shape of closing or opening an extratube gap while being unfolded or folded along the flow direction of urine flowing along the extratube gap and optimizing the shape and arrangement structure of the extratube backflow prevention mechanism.

In accordance with the antireflux ureteral stent according to an embodiment of the present disclosure and the method of manufacturing the same, urine normally flowing along the extratube gap can be smoothly guided from the kidneys to the bladder and the flow of urine abnormally flowing along the extratube gap can be stably blocked by manufacturing the extratube backflow prevention mechanism in a shape of closing/opening the extratube gap while being folded or unfolded along the flow direction of urine flowing along the extratube gap. That is, in this embodiment, urine passages can be formed between a canopy member of the extratube backflow prevention mechanism and the ureter while the canopy member is folded by the flow pressure of urine flowing forward when urine flows in a normal direction along the extratube gap, and the urine passages formed between the canopy member of the extratube backflow prevention mechanism and the ureter can be removed while the canopy member is unfolded by the flow pressure of backflowing urine when urine flows in an abnormal direction along the extratube gap.

In accordance with the antireflux ureteral stent according to an embodiment of the present disclosure and the method of manufacturing the same, the extratube backflow prevention mechanism can be conveniently manufactured according to a casting process using a mold and die made for casting according to a 3D printing process, and the extratube backflow prevention mechanism can be smoothly manufactured in a structure with an expanded star-shaped cross section.

In accordance with the antireflux ureteral stent according to an embodiment of the present disclosure and the method of manufacturing the same, a fine concavo-convex pattern is formed on the surfaces of a mold and die in a process of manufacturing the mold and the die in a 3D printing process, so that a roughness pattern corresponding to the fine concavo-convex pattern of the mold and the die can be formed on the surface of the extratube backflow prevention mechanism in a process of manufacturing the extratube backflow prevention mechanism according to a casting process. Accordingly, the extratube backflow prevention mechanism can be disposed on the outer side surface of the stent body, thereby effectively preventing urine from flowing away from the stent body due to flow pressure, external shock, etc.

The embodiments of the present disclosure have been described with reference to specific details such as specific components and limited examples and drawings as described above. However, this is only provided to facilitate a more general understanding of the present disclosure, and the present disclosure is not limited to the embodiments. Various modifications and variations can be made from these descriptions by those with ordinary knowledge in the field to which the present disclosure belongs. Therefore, the idea of the present disclosure should not be limited to the described embodiments, and not only the accompanying claims, but also all things that are equivalent or equivalent to the claims will fall within the scope of the idea of the present disclosure idea.

### DESCRIPTION OF SYMBOLS

100: antireflux ureteral stent
200: stent body
210: body part
212: installation area
214: installation groove
220: bladder insertion part
230: kidney insertion part
300: extratube backflow prevention mechanism
302: urine passages
304: urine inlet
306: roughness pattern
310: fixation member
320: support beam member
330: canopy member
400: mold
410: die
420: heater
430: immersion liquid
500: performance test device
10: kidney
20: bladder
30: ureter
40: extratube gap
UR: urine
D1: forward direction
D2: reverse direction
P1, P2, P3: first to third positions

What is claimed is:

1. An antireflux ureteral stent, comprising:
a hollow tube-shaped stent body inserted into ureter to guide urine from kidneys to bladder and provided with a flow path for flow of the urine thereinside; and
an extratube backflow prevention mechanism provided in an umbrella shape, made of a flexible material, on an outer side surface of the stent body to be unfolded or folded along a flow direction of the urine and configured to prevent backflow of the urine while being unfolded by flow pressure of the urine when the urine backflows along an extratube gap formed between the stent body and the ureter,
wherein the extratube backflow prevention mechanism is formed in a star-shaped cross-section with a plurality of vertices centered on the stent body and provided with a star-shaped cross section that expands toward the bladder along a longitudinal direction of the stent body.

2. The antireflux ureteral stent according to claim 1, wherein the size of the star-shaped cross section of the extratube backflow prevention mechanism is reduced while urine passages for passing the urine are formed between the vertices of the star-shaped cross section as spaces between the vertices of the star-shaped cross section are folded into a furrow shape when a flow direction of the urine is a forward direction, and increases while the urine passages formed between the vertices of the star-shaped cross section are removed as spaces between the vertices of the star-shaped cross section expand and spread when a flow direction of the urine is a reverse direction.

3. The antireflux ureteral stent according to claim 2, wherein the extratube backflow prevention mechanism comprises:
a fixation member fixed to an outer side surface of the stent body;
a plurality of support beam members configured to expand long from the fixation member to the bladder to form the vertices of the star-shaped cross section of the stent body and formed in an inclined structure far away from the stent body as approaching the bladder; and
a plurality of canopy members respectively provided between the support beam members to shield spaces between the support beam members.

4. The antireflux ureteral stent according to claim 3, wherein the extratube backflow prevention mechanism is detachably mounted on an outer side surface of the stent body, and
the fixation member is formed in a tubular shape surrounding the outer side surface of the stent body, and an installation groove for inserting and mounting the fixation member is provided in a recessed structure along a perimeter of the outer side surface of the stent body.

5. The antireflux ureteral stent according to claim 3, wherein the extratube backflow prevention mechanism is integrally formed with the stent body, and
the fixation member is connected to the outer side surface of the stent body in an integrated structure.

6. The antireflux ureteral stent according to claim 3, wherein the support beam members are arranged to be spaced apart from each other at regular intervals along a perimeter of the fixation member and formed to be thicker than the canopy member to stably support the canopy members, and
the canopy members are folded in a furrow shape toward the stent body according to flow pressure of the urine when a flow direction of the urine is a forward direction, and unfolded while expanding in an opposite direction of the stent body according to flow pressure of the urine when the flow direction of the urine is a reverse direction.

7. The antireflux ureteral stent according to claim 6, wherein the support beam members and the canopy members are formed in a funnel structure expanding along a longitudinal direction of the stent body toward the bladder side to form a urine inlet, into which the urine is introduced when the urine flows back, in an always open state.

8. The antireflux ureteral stent according to claim 6, wherein five support beam members are provided to radially extend from the fixation member, and
the canopy members are respectively disposed between the support beam members and provided in a shape convexly curved toward the stent body such that they can be folded toward the outer side surface of the stent body.

9. The antireflux ureteral stent according to claim 3, wherein a bladder-side end of the stent body is disposed inside one end of the ureter connected to the bladder, and a kidney-side end of the stent body is disposed inside another end of the ureter connected to the kidneys, and
at least one extratube backflow prevention mechanism is disposed on the bladder-side end of the stent body.

10. The antireflux ureteral stent according to claim 9, wherein a single extratube backflow prevention mechanism is disposed at a part connected to the bladder in the bladder-side end of the stent body.

11. The antireflux ureteral stent according to claim 1, wherein the extratube backflow prevention mechanism is manufactured in an integrated structure through a casting process using a mold and a die, and
the mold and the die are manufactured through a 3D printing process.

12. The antireflux ureteral stent according to claim 11, wherein a cavity is formed inside the mold and the die in a shape corresponding to the extratube backflow prevention mechanism, and a fine concavo-convex pattern corresponding to a stacking pattern of filaments is formed on a surface of the cavity in a process of manufacturing the mold and the die through a 3D printing process, and
a roughness pattern corresponding to the fine concavo-convex pattern is formed on a surface of the extratube backflow prevention mechanism in a casting process using the mold and the die.

13. A method of manufacturing an antireflux ureteral stent, the method comprising:
manufacturing a mold and a die used in a casting process of an extratube backflow prevention mechanism using a 3D printing process; and
manufacturing the extratube backflow prevention mechanism according to a casting process using the mold and the die.

14. The method according to claim 13, wherein the manufacturing of the extratube backflow prevention mechanism comprises:
pouring a liquid elastomer material into the mold;
coupling the die to the mold after degassing the elastomer material;
thermally curing the elastomer material disposed in a cavity between the mold and the die with a heater;
immersing the extratube backflow prevention mechanism, the mold and the die in an immersion liquid to peel the extratube backflow prevention mechanism from a surface of the cavity when the elastomer material is cured to form the extratube backflow prevention mechanism;
taking the extratube backflow prevention mechanism, the mold and the die out of the immersion liquid, and then separating the mold and the die: and
removing the extratube backflow prevention mechanism from an inside of the mold to complete manufacturing of the extratube backflow prevention mechanism.

15. The method according to claim 14, wherein ecoflex is provided as the elastomer material, and
acetone is provided as the immersion liquid.

16. The method according to claim 14, wherein in the thermally heating of the heater, the elastomer material is heated at 45 to 50° C. for 15 to 25 minutes with the heater to cure the elastomer material, and
in the immersing of the immersion liquid, the extratube backflow prevention mechanism, the mold and the die are immersed in the immersion liquid for 10 to 14 hours.

17. The method according to claim 13, wherein in the manufacturing of the mold and the die, the mold and the die are respectively manufactured using filaments made of polylactic acid (PLA) with a 3D printer by 3D printing method.

18. The method according to claim 17, wherein a fine concavo-convex pattern is formed according to a stacking pattern of the filaments on surfaces of the mold and the die, and
in the manufacturing of the extratube backflow prevention mechanism, a roughness pattern is formed on a surface of the extratube backflow prevention mechanism in a shape corresponding to the fine concavo-convex pattern of the mold and the die.
